(19) United States
(12) Patent Application Publication
(10) Pub. No.: US 2005/0106705 A1
(43) Pub. Date: May 19, 2005

(54) Methods and compositions for treating hyperlipemia

(76) Inventor: Ling Yuk Cheung, New Territories (HK)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/717,275

(22) Filed: Nov. 18, 2003

(51) Int. Cl. A61K 35/70; A61K 35/72; C12N 1/18

(52) U.S. Cl. 435/254.21; 424/195.16

(57) ABSTRACT

Compositions comprising a plurality of yeast cells, wherein said plurality of yeast cells are characterized by their ability to treat hyperlipemia (e.g., regulate triglyceride and/or cholesterol levels) in a subject as a result of having been cultured in the presence of an alternating electric field having a specific frequency and a specific field strength. Also included are methods of making and using such compositions.

### FIELD OF THE INVENTION

The invention relates to compositions that can ameliorate or prevent hyperlipemia and are useful as dietary supplements (e.g., health drinks) or medication. These compositions contain yeast cells obtainable by growth in electromagnetic fields with specific frequencies and field strengths.

### BACKGROUND OF THE INVENTION

Hyperlipemia is a state of higher than normal blood concentration of lipid components, such as cholesterols, neutral fats, phospholipids or free fatty acids that are in the form of water-soluble lipoproteins. Hyperlipemia is caused by abnormal lipoprotein metabolism. A prolonged hyerlipmic status has been linked to diseases of the circulatory system such as arteriosclerosis, myocardial infarction, angina pectoris, cerebral infarction, apoplexy, coronary diseases, cerebrovascular disorders, high blood pressure, and obesity.

Conventional therapeutic agents for the treatment of hyperlipemia include clofibrate, clinofibrate, phenofibrate, bezafibrate and the like, probucol and nicotinic acid. However, the clofibrate-type drugs are accompanied by adverse side effects such as formation of gallstone, muscular disorders, hepatic dysfunctions and gastrointestinal disorders. Moreover, these drugs must be administered in a large quantity to obtain a certain clinical effect. As a result, severe side effects are frequently observed. There remains a need for an effective agent for treating hyperlipemia.

### SUMMARY OF THE INVENTION

This invention is based on the discovery that certain yeast cells can be activated by electromagnetic fields having specific frequencies and field strengths to produce substances useful in treating hyperlipidemia (for example, regulating triglyceride and/or cholesterol levels). Compositions comprising these activated yeast cells can therefore be used as medication or as dietary supplements, in the form of health drinks or dietary pills (tablets or powder). For instance, these compositions can be used to alleviate (e.g., lower) high blood lipid concentration in a hyperlipmic human, or to prevent or postpone the onset of hyperlipemia in a high risk individual (e.g., someone predisposed to hyperlipemia because of his genetic background or life style).

This invention embraces a composition comprising a plurality of yeast cells that have been cultured in an alternating electric field having a frequency in the range of about 7000 to 13000 MHz (e.g., 7500-8000, 10000-10500, and/or 12400-12800 MHz) and a field strength in the range of about 200 to 450 mV/cm (e.g., 220-240, 270-290, 300-330, 310-340, 320-350, 340-370, 350-380, 360-390, 370-400, 390-430, and/or 420-450 mV/cm). The yeast cells are cultured for a period of time sufficient to activate said plurality of yeast cells to treat hyperlipemia in a subject. In one embodiment, the frequency and/or the field strength of the alternating electric field can be altered within the aforementioned ranges during said period of time. In other words, the yeast cells are exposed to a series of electromagnetic fields. An exemplary period of time is about 80-150 hours (e.g., 100-130 hours).

Also included in this invention is a composition comprising a plurality of yeast cells that have been cultured under acidic conditions in an alternating electric field having a frequency in the range of about 12000 to 13000 MHz (e.g., 12400-12800 MHz) and a field strength in the range of about 200 to 450 mV/cm (e.g., 310-340 and/or 350-380 mV/cm). In one embodiment, the yeast cells are exposed to a series of electromagnetic fields. An exemplary period of time is about 40-80 hours (e.g., 50-66 hours).

Yeast cells that can be included in this composition can be derived from parent strains publically available from the China General Microbiological Culture Collection Center (“CGMCC”), China Committee for Culture Collection of Microorganisms, Institute of Microbiology, Chinese Academy of Sciences, Haidian, P.O. Box 2714, Beijing, 100080, China. Useful yeast species include, but are not limited to, those commonly used in food and pharmaceutical industries, such as Saccharomyces cerevisiae (e.g., Hansen and Hansen Var. ellipsoideus), Saccharomyces carlsbergensis, Saccharomyces rouxii, Saccharomyces sake, Saccharomyces uvarum, Saccharomyces sp., Schizosaccharomyces pombe, Rhodotorula glutinis and Rhodotorula aurantiaca. For instance, the yeast cells can be of the strain Saccharomyces cerevisiae Hansen AS2.560 or ACCC2038, Saccharomyces sp. AS2.3 11, Schizosaccharomyces pombe Lindner AS2.259, Saccharomyces sake Yabe ACCC2045, Saccharomyces uvarum Beijer IFFI1036, Saccharomyces rouxii Boutroux AS2.371, Saccharomyces cerevisiae Hansen Var. ellipsoideus AS2.559, Saccharomyces carlsbergensis Hansen AS2.440, or Rhodotorula glutinis (Fresenius) Harrison AS2.704. Other useful yeast strains are illustrated in Table 1.

This invention further embraces a composition comprising a plurality of yeast cells, wherein said plurality of yeast cells have been activated to treat hyperlipemia in a subject. Included in this invention are also methods of making the above compositions.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. The materials, methods, and examples are illustrative only and not intended to be limiting. Throughout this specification and claims, the word “comprise,” or variations such as “comprises” or “comprising” will be understood to imply the inclusion of a stated integer or group of integers but not the exclusion of any other integer or group of integers. A subject includes a human and veterinary subject.

Other features and advantages of the invention will be apparent from the following detailed description, and from the claims.

### BRIEF DESCRIPTION OF THE DRAWINGS

FIG. 1 is a schematic diagram showing an exemplary apparatus for activating yeast cells using electromagnetic fields. 1: yeast culture; 2: container; 3: power supply.

FIG. 2 is a schematic diagram showing an exemplary apparatus for making yeast compositions of the invention. The apparatus comprises a signal generator (such as models 83721B and 83741A manufactured by HP) and interconnected containers A, B and C.

### DETAILED DESCRIPTION OF THE INVENTION

This invention is based on the discovery that certain yeast strains can be activated by electromagnetic fields (“EMF”) having specific frequencies and field strengths to become highly efficient in producing substances that lower triglyceride and cholesterol levels in a subject. Compositions containing these activated yeast cells are useful in the treatment of hyperlipemia. Yeast compositions containing activated yeast cells can be used as medication or as dietary supplements, in the form of health drinks or dietary pills (tablets or powder).

Since the activated yeast cells contained in the yeast compositions have been cultured to endure acidic conditions (pH 2.5-4.2), these cells can survive the gastric environment and pass on to the intestines. Once in the intestines, the yeast cells are ruptured by various digestive enzymes, and the anti-hyperlipemic substances are released and readily absorbed.

### I. YEAST STRAINS USEFUL IN THE INVENTION

The types of yeasts useful in this invention include, but are not limited to, yeasts of the genera Saccharomyces, Schizosaccharomyces and Rhodotorula.

Exemplary species within the above-listed genera include, but are not limited to, those illustrated in Table 1. Yeast strains useful for this invention can be obtained from laboratory cultures, or from publically accessible culture depositories, such as CGMCC and the American Type Culture Collection, 10801 University Boulevard, Manassas, VA 20110-2209. Non-limiting examples of useful strains (with accession numbers of CGMCC) are Saccharomyces cerevisiae Hansen AS2.560 and ACCC2038, Saccharomyces sp. AS2.311, Schizosaccharomyces pombe Lindner AS2.259, Saccharomyces sake Yabe ACCC2045, Saccharomyces uvarum Beijer IFF11036, Saccharomyces rouxii Boutroux AS2.371, Saccharomyces cerevisiae Hansen Var. ellipsoideus AS2.559, Saccharomyces carlsbergensis Hansen AS2.440, and Rhodotorula glutinis (Fresenius) Harrison AS2.704. Other useful yeast strains are illustrated in Table 1.

The preparation of the yeast compositions of this invention is not limited to starting with a pure strain of yeast. A yeast composition of the invention may be produced by culturing a mixture of yeast cells of different species or strains. The ability of any activated species or strain of yeasts to treat hyperlipemia can be readily tested by methods known in the art. See, for instance, Examples 1 and 2.

TABLE 1
Exemplary Yeast Strains
Saccharomyces cerevisiae Hansen

| | | | | |
|---|---|---|---|---|
| ACCC2034 | ACCC2035 | ACCC2036 | ACCC2037 | ACCC2038 |
| ACCC2039 | ACCC2040 | ACCC2041 | ACCC2042 | AS2.1 |
| AS2.4 | AS2.11 | AS2.14 | AS2.16 | AS2.56 |
| AS2.69 | AS2.70 | AS2.93 | AS2.98 | AS2.101 |
| AS2.109 | AS2.110 | AS2.112 | AS2.139 | AS2.173 |
| AS2.174 | AS2.182 | AS2.196 | AS2.242 | AS2.336 |
| AS2.346 | AS2.369 | AS2.374 | AS2.375 | AS2.379 |
| AS2.380 | AS2.382 | AS2.390 | AS2.393 | AS2.395 |
| AS2.396 | AS2.397 | AS2.398 | AS2.399 | AS2.400 |
| AS2.406 | AS2.408 | AS2.409 | AS2.413 | AS2.414 |
| AS2.415 | AS2.416 | AS2.422 | AS2.423 | AS2.430 |
| AS2.431 | AS2.432 | AS2.451 | AS2.452 | AS2.453 |
| AS2.458 | AS2.460 | AS2.463 | AS2.467 | AS2.486 |
| AS2.501 | AS2.502 | AS2.503 | AS2.504 | AS2.516 |
| AS2.535 | AS2.536 | AS2.558 | AS2.560 | AS2.561 |
| AS2.562 | AS2.576 | AS2.593 | AS2.594 | AS2.614 |
| AS2.620 | AS2.628 | AS2.631 | AS2.666 | AS2.982 |
| AS2.1190 | AS2.1364 | AS2.1396 | IFFI1001 | IFFI1002 |
| IFFI1005 | IFFI1006 | IFFI1008 | IFFI1009 | IFFI1010 |
| IFFI1012 | IFFI1021 | IFFI1027 | IFFI1037 | IFFI1042 |
| IFFI1043 | IFFI1045 | IFFI1048 | IFFI1049 | IFFI1050 |
| IFFI1052 | IFFI1059 | IFFI1060 | IFFI1062 | IFFI1063 |
| IFFI1202 | IFFI1203 | IFFI1206 | IFFI1209 | IFFI1210 |
| IFFI1211 | IFFI1212 | IFFI1213 | IFFI1214 | IFFI1215 |
| IFFI1220 | IFFI1221 | IFFI1224 | IFFI1247 | IFFI1248 |
| IFFI1251 | IFFI1270 | IFFI1277 | IFFI1287 | IFFI1289 |
| IFFI1290 | IFFI1291 | IFFI1292 | IFFI1293 | IFFI1297 |
| IFFI1300 | IFFI1301 | IFFI1302 | IFFI1307 | IFFI1308 |
| IFFI1309 | IFFI1310 | IFFI1311 | IFFI1331 | IFFI1335 |
| IFFI1336 | IFFI1337 | IFFI1338 | IFFI1339 | IFFI1340 |
| IFFI1345 | IFFI1348 | IFFI1396 | IFFI1397 | IFFI1399 |
| IFFI1411 | IFFI1413 | IFFI1441 | IFFI1443 | |

Saccharomyces cerevisiae Hansen Var. ellipsoideus (Hansen) Dekker

| | | | | |
|---|---|---|---|---|
| ACCC2043 | AS2.2 | AS2.3 | AS2.8 | AS2.53 |
| AS2.163 | AS2.168 | AS2.483 | AS2.541 | AS2.559 |
| AS2.606 | AS2.607 | AS2.611 | AS2.612 | |

Saccharomyces chevalieri Guilliermond

| | |
|---|---|
| AS2.131 | AS2.213 |

Saccharomyces delbrueckii

AS2.285

Saccharomyces delbrueckii Lindner ver. mongolicus (Saito) Lodder et van Rij

| | |
|---|---|
| AS2.209 | AS2.1157 |

Saccharomyces exiguous Hansen

| | |
|---|---|
| AS2.349 | AS2.1158 |

Saccharomyces fermentati (Saito) Lodder et van Rij

| | |
|---|---|
| AS2.286 | AS2.343 |

Saccharomyces logos van laer et Denamur ex Jorgensen

| | | |
|---|---|---|
| AS2.156 | AS2.327 | AS2.335 |

Saccharomyces mellis (Fabian et Quinet) Lodder et kreger van Rij

AS2.195

Saccharomyces mellis Microellipsoides Osterwalder

AS2.699

Saccharomyces oviformis Osteralder

AS2.100

Saccharomyces rosei (Guilliermond) Lodder et Kreger van Rij

AS2.287

Saccharomyces rouxii Boutroux

| | | | |
|---|---|---|---|
| AS2.178 | AS2.180 | AS2.370 | AS2.371 |

Saccharomyces sake Yabe

ACCC2045

Candida arborea

AS2.566

Candida lambica (Lindner et Genoud) van. Uden et Buckley

AS2.1182

Candida krusei (Castellani) Berkhout

AS2.1045

Candida lipolytica (Harrison) Diddens et Lodder

| | | | | |
|---|---|---|---|---|
| AS2.1207 | AS2.1216 | AS2.1220 | AS2.1379 | AS2.1398 |
| AS2.1399 | AS2.1400 | | | |

Candida parapsilosis (Ashford) Langeron et Talice Var. intermedia Van Rij et Verona

AS2.491

Candida parapsilosis (Ashford) Langeron et Talice

AS2.590

Candida pulcherrima (Lindner) Windisch

AS2.492

Candida rugousa (Anderson) Diddens et Lodder

| | | | | |
|---|---|---|---|---|
| AS2.511 | AS2.1367 | AS2.1369 | AS2.1372 | AS2.1373 |
| AS2.1377 | AS2.1378 | AS2.1384 | | |

Candida tropicalis (Castellani) Berkhout

| | | | | |
|---|---|---|---|---|
| ACCC2004 | ACCC2005 | ACCC2006 | AS2.164 | AS2.402 |
| AS2.564 | AS2.565 | AS2.567 | AS2.568 | AS2.617 |
| AS2.637 | AS2.1387 | AS2.1397 | | |

Candida utilis Henneberg Lodder et Kreger Van Rij

| | | |
|---|---|---|
| AS2.120 | AS2.281 | AS2.1180 |

Crebrothecium ashbyii (Guillermond)
Routein (Eremothecium ashbyii Guilliermond)

| | | |
|---|---|---|
| AS2.481 | AS2.482 | AS2.1197 |

Geotrichum candidum Link

| | | | | |
|---|---|---|---|---|
| ACCC2016 | AS2.361 | AS2.498 | AS2.616 | AS2.1035 |
| AS2.1062 | AS2.1080 | AS2.1132 | AS2.1175 | AS2.1183 |

Hansenula anomala (Hansen)H et P sydow

| | | | | |
|---|---|---|---|---|
| ACCC2018 | AS2.294 | AS2.295 | AS2.296 | AS2.297 |
| AS2.298 | AS2.299 | AS2.300 | AS2.302 | AS2.338 |
| AS2.339 | AS2.340 | AS2.341 | AS2.470 | AS2.592 |
| AS2.641 | AS2.642 | AS2.782 | AS2.635 | AS2.794 |

Hansenula arabitolgens Fang

AS2.887

Hansenula jadinii (A. et R Sartory Weill et Meyer) Wickerham

ACCC2019

Hansenula saturnus (Klocker) H et P sydow

ACCC2020

Hansenula schneggii (Weber) Dekker

AS2.304

Hansenula subpelliculosa Bedford

| | | | | |
|---|---|---|---|---|
| AS2.740 | AS2.760 | AS2.761 | AS2.770 | AS2.783 |
| AS2.790 | AS2.798 | AS2.866 | | |

Kloeckera apiculata (Reess emend. Klocker) Janke

| | | | | |
|---|---|---|---|---|
| ACCC2022 | ACCC2023 | AS2.197 | AS2.496 | AS2.714 |
| ACCC2021 | AS2.711 | | | |

Lipomycess starkeyi Lodder et van Rij

| | |
|---|---|
| AS2.1390 | ACCC2024 |

Pichia farinosa (Lindner) Hansen

| | | | | |
|---|---|---|---|---|
| ACCC2025 | ACCC2026 | AS2.86 | AS2.87 | AS2.705 |
| AS2.803 | | | | |

Pichia membranaefaciens Hansen

| | | | |
|---|---|---|---|
| ACCC2027 | AS2.89 | AS2.661 | AS2.1039 |

Rhodosporidium toruloides Banno

ACCC2028

Rhodotorula glutinis (Fresenius) Harrison

| | | | | |
|---|---|---|---|---|
| AS2.2029 | AS2.280 | ACCC2030 | AS2.102 | AS2.107 |
| AS2.278 | AS2.499 | AS2.694 | AS2.703 | AS2.704 |
| AS2.1146 | | | | |

Rhodotorula minuta (Saito) Harrison

AS2.277

Rhodotorula rubar (Demme) Lodder

| | | | | |
|---|---|---|---|---|
| AS2.21 | AS2.22 | AS2.103 | AS2.105 | AS2.108 |
| AS2.140 | AS2.166 | AS2.167 | AS2.272 | AS2.279 |
| AS2.282 | ACCC2031 | | | |

Rhodotorula aurantiaca (Saito) Lodder

| | | | | |
|---|---|---|---|---|
| AS2.102 | AS2.107 | AS2.278 | AS2.499 | AS2.694 |
| AS2.703 | AS2.1146 | | | |

Saccharomyces carlsbergensis Hansen

| | | | | |
|---|---|---|---|---|
| AS2.113 | ACCC2032 | ACCC2033 | AS2.312 | AS2.116 |
| AS2.118 | AS2.121 | AS2.132 | AS2.162 | AS2.189 |
| AS2.200 | AS2.216 | AS2.265 | AS2.377 | AS2.417 |
| AS2.420 | AS2.440 | AS2.441 | AS2.443 | AS2.444 |
| AS2.459 | AS2.595 | AS2.605 | AS2.638 | AS2.742 |
| AS2.745 | AS2.748 | AS2.1042 | | |

Saccharomyces uvarum Beijer

| | | | | |
|---|---|---|---|---|
| IFFI1023 | IFFI1032 | IFFI1036 | IFFI1044 | IFFI1072 |
| IFFI1205 | IFFI1207 | | | |

Saccharomyces willianus Saccardo

| | | | | |
|---|---|---|---|---|
| AS2.5 AS2.7 | AS2.119 | AS2.152 | AS2.293 | |
| AS2.381 | AS2.392 | AS2.434 | AS2.614 | AS2.1189 |

Saccharomyces sp.

AS2.311

Saccharomycodes ludwigii Hansen

| | | |
|---|---|---|
| ACCC2044 | AS2.243 | AS2.508 |

Saccharomycodes sinenses Yue

AS2.1395

Schizosaccharomyces octosporus Beijerinck

| | |
|---|---|
| ACCC2046 | AS2.1148 |

Schizosaccharomyces pombe Lindner

| | | | | |
|---|---|---|---|---|
| ACCC2047 | ACCC2048 | AS2.214 | AS2.248 | AS2.249 |
| AS2.255 | AS2.257 | AS2.259 | AS2.260 | AS2.274 |
| AS2.994 | AS2.1043 | AS2.1149 | AS2.1178 | IFFI1056 |

Sporobolomyces roseus Kluyver et van Niel

| | | | | |
|---|---|---|---|---|
| ACCC2049 | ACCC2050 | AS2.19 | AS2.962 | AS2.1036 |
| ACCC2051 | AS2.261 | AS2.262 | | |

Torulopsis candida (Saito) Lodder

| | |
|---|---|
| AS2.270 | ACCC2052 |

Torulopsis famta (Harrison) Lodder et van Rij

| | |
|---|---|
| ACCC2053 | AS2.685 |

Torulopsis globosa (Olson et Hammer) Lodder et van Rij

| | |
|---|---|
| ACCC2054 | AS2.202 |

Torulopsis inconspicua Lodder et Kreger van Rij

AS2.75

Trichosporon behrendii Lodder et Kreger van Rij

| | |
|---|---|
| ACCC2056 | AS2.1193 |

Trichosporon capitatum Diddens et Lodder

| | |
|---|---|
| ACCC2056 | AS2.1385 |

Trichosporon cutaneum (de Beurm et al.) Ota

| | | | | |
|---|---|---|---|---|
| ACCC2057 | AS2.25 | AS2.570 | AS2.571 | AS2.1374 |

Wickerhamia fluorescens (Soneda) Soneda

| | |
|---|---|
| ACCC2058 | AS2.1388 |

### II. APPLICATION OF ELECTROMAGNETIC FIELDS

An electromagnetic field useful in this invention can be generated and applied by various means well known in the art. For instance, the EMF can be generated by applying an alternating electric field or an oscillating magnetic field.

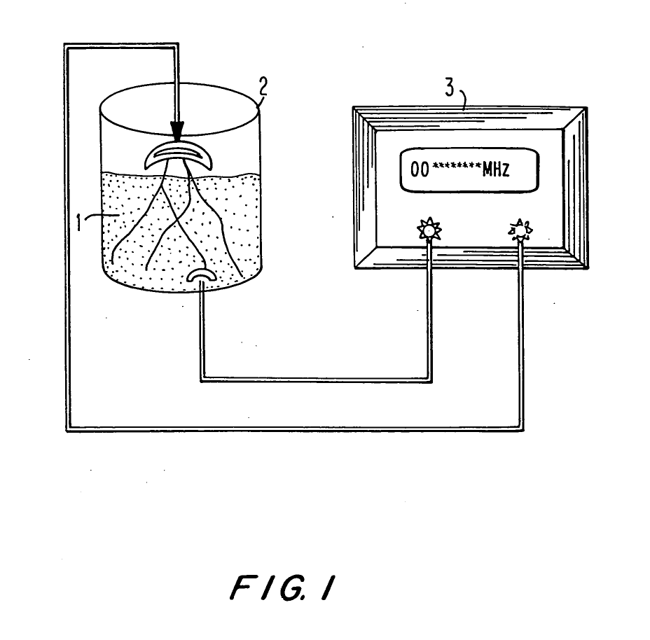

Alternating electric fields can be applied to cell cultures through electrodes in direct contact with the culture medium, or through electromagnetic induction. See, e.g., FIG. 1. Relatively high electric fields in the medium can be generated using a method in which the electrodes are in contact with the medium. Care must be taken to prevent electrolysis at the electrodes from introducing undesired ions into the culture and to prevent contact resistance, bubbles, or other features of electrolysis from dropping the field level below that intended. Electrodes should be matched to their environment, for example, using Ag—AgCl electrodes in solutions rich in chloride ions, and run at as low a voltage as possible. For general review, see Goodman et al., Effects of EMF on Molecules and Cells, International Review of Cytology, A Survey of Cell Biology, Vol. 158, Academic Press, 1995.

The EMFs useful in this invention can also be generated by applying an oscillating magnetic field. An oscillating magnetic field can be generated by oscillating electric currents going through Helmholtz coils. Such a magnetic field in turn induces an electric field.

The frequencies of EMFs useful in this invention range from about 7000-13000 MHz (e.g., 7500-8000, 10000-10500, and/or 12400-12800 MHz). Exemplary frequencies are 7858, 7873, 10072, 12623, and 12642 MHz. The field strength of the electric field useful in this invention ranges from about 200 to 450 mV/cm (e.g., 220-240, 270-290, 300-330, 310-340, 320-350, 340-370, 350-380, 360-390, 370-400, 390-430, and/or 420-450 mV/cm). Exemplary field strengths are 228, 276, 284, 305, 317, 320, 332, 346, 363, 374, 384, and 407 mV/cm.

When a series of EMFs are applied to a yeast culture, the yeast culture can remain in the same container while the same set of EMF generator and emitters is used to change the frequency and/or field strength. The EMFs in the series can each have a different frequency or a different field strength; or a different frequency and a different field strength. Such frequencies and field strengths are preferably within the above-described ranges. Although any practical number of EMFs can be used in a series, it may be preferred that the yeast culture be exposed to a total of 2, 3, 4, 5, 6, 7, 8, 9, 10, 11, 12, 13 or more EMFs in a series. In one embodiment, the yeast culture is exposed to a series of EMFs, wherein the frequency of the electric field is alternated in the range of about 7500-8000, 10000-10500, and 12400-12800 MHz.

Although the yeast cells can be activated after even a few hours of culturing in the presence of an EMF, it may be preferred that the activated yeast cells be allowed to multiply and grow in the presence of the EMF(s) for a total of 80-150 hours (e.g., 100-130 hours).

FIG. 1 illustrates an exemplary apparatus for generating alternating electric fields. An electric field of a desired frequency and intensity is generated by an AC source (3) capable of generating an alternating electric field, preferably in a sinusoidal wave form, in the frequency range of 10 to 20,000 MHz. Signal generators capable of generating signals with a narrower frequency range can also be used. If desirable, a signal amplifier can also be used to increase the output. The activation container (2) can be made from non-conductive material, for example, plastics, glass steel, ceramic, and combinations thereof. The wire connecting the activation container (2) and the signal generator (3) is preferably a high frequency coaxial cable with a transmission frequency of at least 30 GHz.

The alternating electric field can be applied to the culture by a variety of means, including placing the yeast culture (1) in close proximity to the signal emitters such as a metal wire or tube capable of transmitting EMFs. The metal wire or tube can be made of red copper, and be placed inside the container (2), reaching as deep as 3-30 cm. For example, if the fluid in the container (2) has a depth of 15-20 cm, 20-30 cm, 30-50 cm, 50-70 cm, 70-100 cm, 100-150 cm or 150-200 cm, the metal wire can be 3-5 cm, 5-7 cm, 7-10 cm, 10-15 cm, 15-20 cm, 20-30 cm and 25-30 cm from the bottom of the container (2), respectively. The number of electrode wires used depends on the volume of the culture as well as the diameter of the wires. The number of metal wires/tubes used can be from 1 to 10 (e.g., 2 to 3). It is recommended, though not mandated, that for a culture having a volume up to 10 L, metal wires/tubes having a diameter of 0.5 to 2.0 mm be used. For a culture having a volume between 10 L and 100 L, metal wires/tubes having a diameter of 3.0 to 5.0 mm can be used. For a culture having a volume in the range of 100-1000 L, metal wires/tubes having a diameter of 6.0 to 15.0 mm can be used. For a culture having a volume greater than 1000 L, metal wires/tubes having a diameter of 20.0 to 25.0 mm can be used.

In one embodiment, the electric field is applied by electrodes submerged in the culture (1). In this embodiment, one of the electrodes can be a metal plate placed on the bottom of the container (2), and the other electrode can comprise a plurality of electrode wires evenly distributed in the culture (1) so as to achieve even distribution of the electric field energy. The number of electrode wires used depends on the volume of the culture as well as the diameter of the wires.

### III. CULTURE MEDIA

Culture media useful in this invention contain sources of nutrients assimilable by yeast cells. Complex carbon-containing substances in a suitable form, such as carbohydrates (e.g., sucrose, glucose, fructose, dextrose, maltose, xylose, cellulose, starches, etc.) and coal, can be the carbon sources for yeast cells. The exact quantity of the carbon sources utilized in the medium can be adjusted in accordance with the other ingredients of the medium. In general, the amount of carbohydrates varies between about 0.1% and 10% by weight of the medium and preferably between about 0.1% and 5% (e.g., about 2%). These carbon sources can be used individually or in combination. Amino acid-containing substances in suitable form (e.g., beef extract and peptone) can also be added individually or in combination. In general, the amount of amino acid containing substances varies between about 0.1% and 0.5% by weight of the medium and preferably between about 0.1% and 0.3% (e.g., about 0.25%). Among the inorganic salts which can be added to the culture medium are the customary salts capable of yielding sodium, potassium, calcium, phosphate, sulfate, carbonate, and like ions. Non-limiting examples of nutrient inorganic salts are (NH₄)₂HPO₄, KH₂PO₄, K₂HPO₄, CaCO₃, MgSO₄, NaCl, and CaSO₄.

### IV. ELECTROMAGNETIC ACTIVATION OF YEAST CELLS

To activate or enhance the ability of yeast cells to produce substances beneficial for the treatment of hyperlipemia (e.g., lowering of triglyceride and/or cholesterol levels), these cells can be activated by being cultured in an appropriate medium under sterile conditions at 20° C.-38° C., preferably at 28-32° C. (e.g., 30° C.) for a sufficient amount of time, e.g., 100-200 hours (e.g., 147-171 hours), in an alternating electric field or a series of alternating electric fields as described above.

An exemplary culture medium is made by mixing 950 ml of distilled water with 20 g of sucrose, 20 μg of vitamin B₁₂, 40 μg of vitamin B₆, 100 μg of vitamin E, 50 ml of fetal bovine serum, 0.20 g of KH₂PO₄, 0.25 g of MgSO₄.7H₂O, 0.3 g of NaCl, 0.2 g of CaSO₄.2H₂O, 4.0 g of CaCO₃.5H₂O, and 2.5 g of peptone.

An exemplary set-up of the culturing process is depicted in FIG. 1. Untreated yeast cells are added to a culture medium at 1×10⁸ cells per 1000 ml of the culture medium. The yeast cells may be Saccharomyces cerevisiae Hansen AS2.560, or may be selected from any of the strains listed in Table 1. An exemplary activation process of the yeast cells involves the following sequence: the yeast cells are grown in the culture medium for 26-30 hours (e.g., 28 hours) at 28-32° C. and then exposed to (1) an alternating electric field having a frequency of 7858 MHz and a field strength in the range of 270-290 mV/cm (e.g., 276 mV/cm) for 14-18 hours (e.g., 16 hours); (2) then to an alternating electric field having a frequency of 7873 MHz and a field strength in the range of 300-330 mV/cm (e.g., 305 mV/cm) for 30-34 hours (e.g., 32 hours); (3) then to an alternating electric field having a frequency of 10072 MHz and a field strength in the range of 300-330 mV/cm (e.g., 317 mV/cm) for 37-41 hours (e.g., 39 hours); (4) then to an alternating electric field having a frequency of 12623 MHz and a field strength in the range of 340-370 mV/cm (e.g., 346 mV/cm) for 27-31 hours (e.g., 29 hours); and (5) finally to an alternating electric field having a frequency of 12642 MHz and a field strength in the range of 270-290 mV/cm (e.g., 284 mV/cm) for 13-17 hours (e.g., 15 hours). The activated yeast cells are then recovered from the culture medium by various methods known in the art, dried (e.g., by lyophilization) and stored at about 4° C. in powder form. The resultant yeast powder preferably contains no less than 10¹⁰ cells/g activated yeast.

Subsequently, the activated yeast cells can be evaluated for their ability to treat hyperlipemia using standard methods known in the art, such as those described in Section VII.

### V. ACCLIMATIZATION OF YEAST CELLS TO THE GASTRIC ENVIRONMENT

Because the activated yeast cells of this invention must pass through the stomach before reaching the small intestine, where the effective components are released from these yeast cells, it is preferred that these yeasts be cultured under acidic conditions so as to acclimatize the cells to the gastric juice. This acclimatization process results in better viability of the yeast cells in the acidic gastric environment.

To achieve this, the yeast powder containing activated yeast cells can be mixed with a highly acidic acclimatizing culture medium at 10 g (containing more than 10¹⁰ activated cells per gram) per 1000 ml. The yeast mixture can then be cultured first in the presence of an alternating electric field having a frequency of 12623 MHz and a field strength in the range of 350-380 mV/cm (e.g., 363 mV/cm) at about 28 to 32° C. for 34-42 hours (e.g., 38 hours). The resultant yeast cells can then be further incubated in the presence of an alternating electric field having a frequency of 12642 MHz and a field strength in the range of 310-340 mV/cm (e.g., 320 mV/cm) at about 28 to 32° C. for 16-24 hours (e.g., 20 hours). The resulting acclimatized yeast cells are then recovered from the culture medium by various methods known in the art and are dried and stored either in powder form (≧10¹⁰ cells/g) at room temperature or in vacuum at 0-4° C.

An exemplary acclimatizing culture medium is made by mixing 700 ml fresh pig gastric juice and 300 ml wild Chinese hawthorn extract. The pH of acclimatizing culture medium is adjusted to 2.5 with 0.1 M hydrochloric acid (HCl) and 0.2 M potassium hydrogen phthalate (C₆H₄(COOK)COOH). The fresh pig gastric juice is prepared as follows. At about 4 months of age, newborn Holland white pigs are sacrificed, and the entire contents of their stomachs are retrieved and mixed with 2000 ml of water under sterile conditions. The mixture is then allowed to stand for 6 hours at 4° C. under sterile conditions to precipitate food debris. The supernatant is collected for use in the acclimatizing culture medium. To prepare the wild Chinese hawthorn extract, 500 g of fresh wild Chinese hawthorn is dried under sterile conditions to reduce water content (≦8%). The dried fruit is then ground (≧20 mesh) and added to 1500 ml of sterile water. The hawthorn slurry is allowed to stand for 6 hours at 4° C. under sterile conditions. The hawthorn supernatant is collected to be used in the acclimatizing culture medium.

### VI. MANUFACTURE OF YEAST COMPOSITIONS

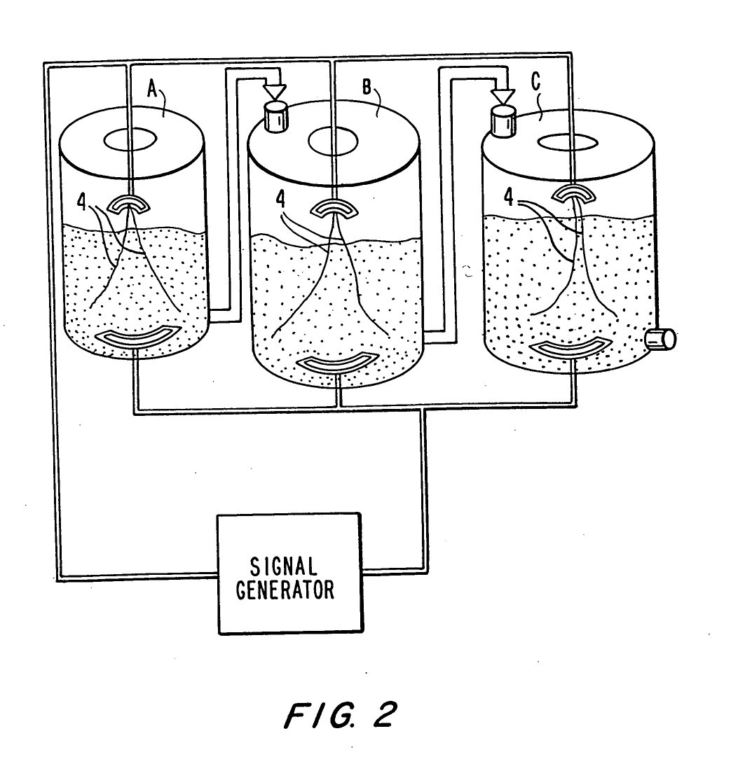

To prepare the yeast compositions of the invention, an apparatus depicted in FIG. 2 or an equivalent thereof can be used. This apparatus includes three containers, a first container (A), a second container (B), and a third container (C), each equipped with a pair of electrodes (4). One of the electrodes is a metal plate placed on the bottom of the containers, and the other electrode comprises a plurality of electrode wires evenly distributed in the space within the container to achieve even distribution of the electric field energy. All three pairs of electrodes are connected to a common signal generator.

The culture medium used for this purpose is a mixed fruit extract solution containing the following ingredients per 1000 L: 300 L of wild Chinese hawthorn extract, 300 L of jujube extract, 300 L of Schisandra chinensis (Turez) Baill seeds extract, and 100 L of soy bean extract. To prepare hawthorn, jujube and Schisandra chinensis (Turez) Baill seeds extracts, the fresh fruits are washed and dried under sterile conditions to reduce the water content to no higher than 8%. One hundred kilograms of the dried fruits are then ground (≧20 mesh) and added to 400 L of sterile water. The mixtures are stirred under sterile conditions at room temperature for twelve hours, and then centrifuged at 1000 rpm to remove insoluble residues. To make the soy bean extract, fresh soy beans are washed and dried under sterile conditions to reduce the water content to no higher than 8%. Thirty kilograms of dried soy beans are then ground into particles of no smaller than 20 mesh, and added to 130 L of sterile water. The mixture is stirred under sterile conditions at room temperature for twelve hours and centrifuged at 1000 rpm to remove insoluble residues. Once the mixed fruit extract solution is prepared, it is autoclaved at 121 ° C. for 30 minutes and cooled to below 40° C. before use.

One thousand grams of the activated yeast powder prepared as described above (Section V, supra) is added to 1000 L of the mixed fruit extract solution, and the yeast solution is transferred to the first container (A) shown in FIG. 2. The yeast cells are then cultured in the presence of an alternating electric field having a frequency of 12623 MHz and a field strength of about 370-400 mV/cm (e.g., 384 mV/cm) at 28-32° C. under sterile conditions for 26 hours. The yeast cells are further incubated in an alternating electric field having a frequency of 12642 MHz and a field strength of 320-350 mV/cm (e.g., 332 mV/cm). The culturing continues for another 12 hours.

The yeast culture is then transferred from the first container (A) to the second container (B) (if need be, a new batch of yeast culture can be started in the now available the first container (A)), and subjected to an alternating electric field having a frequency of 12623 MHz and a field strength of 390-430 mV/cm (e.g., 407 mV/cm) for 24 hours. Subsequently the frequency and field strength of the electric field are changed to 12642 MHz and 360-390 mV/cm (e.g., 374 mV/cm), respectively. The culturing process continues for another 12 hours.

The yeast culture is then transferred from the second container (B) to the third container (C), and subjected to an alternating electric field having a frequency of 12623 MHz and a field strength of 270-290 mV/cm (e.g., 276 mV/cm) for 24 hours. Subsequently the frequency and field strength of the electric field are changed to 12642 MHz and 220-240 mV/cm (e.g., 228 mV/cm), respectively. The culturing continues for another 12 hours.

The yeast culture from the third container (C) can then be packaged into vacuum sealed bottles for use as medicament or dietary supplement, e.g., in the form of health drinks, pills, or powder, etc. If desired, the final yeast culture can also be dried within 24 hours and stored in powder form. The dietary supplement can be taken three to four times daily at 30˜60 ml per dose for a three-month period, preferably 10-30 minutes before meals and at bedtime.

In some embodiments, the compositions of the invention can also be administered intravenously or peritoneally in the form of a sterile injectable preparation. Such a sterile preparation can be prepared as follows. A sterilized health drink composition is first treated under ultrasound (≧18,000 Hz) for 10 minutes and then centrifuged for another 10 minutes. The resulting supernatant is adjusted to pH 7.2-7.4 using 1 M NaOH and subsequently filtered through a membrane (0.22 μm for intravenous injection and 0.45 μm for peritoneal injection) under sterile conditions. The resulting sterile preparation is submerged in a 35-38° C. water bath for 30 minutes before use. In other embodiments, the compositions of the invention may also be formulated with pharmaceutically acceptable carriers to be orally administered in any orally acceptable dosage form including, but not limited to, capsules, tablets, suspensions or solutions.

The yeast compositions of the present invention are derived from yeasts used in food and pharmaceutical industries. The yeast compositions are thus devoid of side effects associated with many pharmaceutical compounds.

### VII. EXAMPLES

In order that this invention be more fully understood, the following examples are set forth. These examples are for the purpose of illustration only and are not to be construed as limiting the scope of the invention in any way.

The activated yeast compositions used in the following examples were prepared as described above, using Saccharomyces cerevisiae Hansen AS2.560 cells, cultured in the presence of an alternating electric field having the electric field frequency and field strength exemplified in the parentheses following the recommended ranges listed in Section IV, supra. Control (i.e., untreated) yeast compositions were those prepared in the same manner as described in Section VI, supra, except that the yeast cells were cultured in the absence of EMFs. All compositions of interest were administered to the animals by intragastric feeding, unless otherwise specified.

### Example 1

### Effects of the Treatment on Cholesterol Levels in Rabbits

To test the ability of the activated yeast compositions to control hyperlipemia, thirty healthy domesticated rabbits (Oryctolagus curiculus, 3.8-4.2 kg, half of them male and half of them female, 18-24 months old) were given regular rabbit feed for four weeks and then were randomly divided into three groups, Group A, B and C. A mixture of 0.2 mg/kg cholesterol+2 ml lard was administered to each rabbit once daily and a composition of interest at a dosage of 1.5 ml/kg was administered twice a day for eight consecutive weeks. Rabbits in Groups A, B and C were given the activated yeast composition, the control yeast composition and saline, respectively, in addition to cholesterol and lard. All rabbits were otherwise maintained the same way.

Prior to the administration of the mixture and the composition of interest, as well as at 2, 4, 6 and 8 weeks after the administration, blood samples were taken from the marginal vein of an ear of each rabbit and centrifuged at 3000 rpm to recover sera. The amount of serum cholesterol was measured by the method described in Table 2 below.

TABLE 2
A Method for Determining Cholesterol Concentration.

| | Contents in Each Test Tube | |
|---|---|---|
| Steps | Standard Tube (ml) | Sample Tube (ml) |
| Mixed Serum, | — | 0.2 |
| Cholesterol Standard | 0.2 | — |
| Solution, (2 mg/ml) | | |
| Glacial Acetic Acid, (ml) | 0.5 | 0.5 |
| 0.9% NaCl, | 0.2 | — |
| and Acetic Anhydride | 4.8 | 5.0 |

Mixed Vigorously for 5 Minutes and Centrifuged for 5 Minutes

| | | |
|---|---|---|
| To the resulting Top | 4.0 | 4.0 |
| Layer, | | |
| Added conc. H₂SO₄ (ml) | 0.2 | 0.2 |

Stirred in a 25° C. water bath for 15 minutes. Readings of the sample and
standard tubes were taken with a spectrophotometer at 630 nm (calibrated
against blank control with distilled water).

A standard curve of cholesterol was established and the concentration of cholesterol in the samples was determined according to the standard curve. The results were shown in Table 3.

TABLE 3
Effects on Cholesterol Concentration

| | Cholesterol (mg/100 ml) | | | | |
|---|---|---|---|---|---|
| Group | Day 1 | 2 weeks | 4 weeks | 6 weeks | 8 weeks |
| A | 83.87 ± | 242.43 ± | 156.55 ± | 98.73 ± | 87.37 ± |
| | 15.42 | 23.64 | 21.34 | 11.78 | 8.79 |
| B | 79.53 ± | 383.65 ± | 643.56 ± | 703.67 ± | 778.76 ± |
| | 14.22 | 29.54 | 65.45 | 64.78 | 86.87 |
| C | 77.46 ± | 377.67 ± | 612.87 ± | 712.32 ± | 823.78 ± |
| | 11.31 | 30.26 | 89.54 | 98.35 | 102.54 |

The above results show that the activated yeast composition was more effective in lowering the cholesterol levels than the control yeast composition.

### Example 2

### Effects of the Treatment on Cholesterol and Triglyceride Levels in Rats

To test the ability of the activated yeast compositions to control hyperlipemia, forty healthy Wistar rats (120-140 g, half of them male and half of them female, 2-4 months old) were randomly divided into four groups, Groups A, B, C and D. Groups A, B and C rats were given high lipid content rat feed and Groups D rats were given regular rat feed for three weeks. A composition of interest at a dosage of 1.5 ml/100 g was then administered to each rat in Groups A, B and C once daily in conjunction with the high lipid content rat feed for another three consecutive weeks. Rats in Group A, B and C were given the activated yeast composition, the control yeast composition and saline, respectively, in addition to the high lipid content rat feed. Rats in Group D were given 1.5 ml/100 g saline and regular rat feed instead. The high lipid content rat feed was prepared by mixing 1% cholesterol, 0.5% bile salt, 0.2% methylthiouracil, 2% lard, 5% soybean, 1% egg, 2% ground dry yolk, 1% fish meal, and 87.3% regular rat feed with water to form a paste.

Twenty-four hours after the last administration of the composition, the rats were given no feed but water for another twelve hours. Blood samples were then taken from the carotid and jugular vein of the rat and centrifuged at 3000 rpm to recover sera. The amount of serum cholesterol was measured by the same method as described in Example 1. The amount of serum triglycerides was measured by the method described in Table 4 below.

TABLE 4
A Method for Determining Triglyceride Concentration.

| | Contents in Each Test Tube | | |
|---|---|---|---|
| | Blank Control | | Sample Tube |
| Steps | Tube (ml) | Standard Tube (ml) | (ml) |
| Mixed Serum, | — | — | 0.2 |
| Triglyceride | — | 0.2 | — |
| Standard Solution, | | | |
| (1.0 mg/ml) | | | |
| Distilled Water | 0.2 | 0.2 | — |
| (ml) | | | |
| with the Extracting | 2.2 | 2.0 | 2.2 |
| Solvent (1:1 v/v n- | | | |
| heptane/i-PrOH) | | | |

Shook vigorously so that triglycerides dissolved in the extracting solvent

| | | | |
|---|---|---|---|
| Added H₂SO₄ | 0.5 | 0.5 | 0.5 |
| (0.04 M) | | | |

Shook vigorously for a few seconds and allowed the mixture to separate
into two layers

| | | | |
|---|---|---|---|
| To the Resulting | 0.4 | 0.4 | 0.4 |
| Top Layer | | | |
| Added 5% KOH | 2.0 | 2.0 | 2.0 |

Stirred in a 65° C. water bath for 5 minutes to allow saponification

| | | | |
|---|---|---|---|
| Added NaIO₄ | 1.0 | 1.0 | 1.0 |
| (0.2 M) | | | |

The mixture was stirred

| | | | |
|---|---|---|---|
| Added | 1.0 | 1.0 | 1.0 |
| Acetylicetone | | | |
| (0.5 M) | | | |

Stirred in a 65° C. water bath for 10 minutes and cooled to room
temperature. Readings of the sample and standard tubes were taken with
a spectrophotometer at 420 nm (calibrated with the blank control tube).

A standard curve of triglyceride was established and the concentration of triglyceride in the samples was determined according to the standard curve. The results were shown in Table 5.

TABLE 5
Effects on Triglyceride and Cholesterol Concentrations

| Group | Cholesterol (mg/100 ml) | Triglyceride (mg/100 ml) |
|---|---|---|
| A | 126.4 ± 43.7 | 87.4 ± 12.6 |
| B | 734.6 ± 214.7 | 223.6 ± 127.8 |
| C | 723.8 ± 221.6 | 213.4 ± 146.7 |
| D | 98.8 ± 36.7 | 59.7 ± 22.6 |

The above results show that the activated yeast composition was more effective in lowering the triglyceride and cholesterol levels than the control yeast composition.

While a number of embodiments of this invention have been set forth, it is apparent that the basic constructions may be altered to provide other embodiments which utilize the compositions and methods of this invention.

1. A composition comprising a plurality of yeast cells, wherein said plurality of yeast cells are characterized by their ability to treat hyperlipemia in a subject, said ability resulting from their having been cultured in the presence of an alternating electric field having a frequency in the range of about 7000 to 13000 MHz and a field strength in the range of about 200 to 450 mV/cm, as compared to yeast cells not having been so cultured.

2. The composition of claim 1, wherein said frequency is in the range of about 7500-8000, 10000-10500, or 12400-12800 MHz.

3. The composition of claim 1, wherein said field strength is in the range of 220-240, 270-290, 300-330, 310-340, 320-350, 340-370, 350-380, 360-390, 370-400, 390-430, or 420-450 mV/cm.

4. The composition of claim 1, wherein said yeast cells are cells of the species Saccharomyces cerevisiae, Saccharomyces carlsbergensis, Saccharomyces rouxii, Saccharomyces sake, Saccharomyces uvarum, Saccharomyces sp., Schizosaccharomyces pombe, Rhodotorula glutinis, or Rhodotorula aurantiaca.

5. The composition of claim 1, wherein said yeast cells are derived from cells of the strain deposited at the China General Microbiological Culture Collection Center with an accession number selected from the group consisting of AS2.560, ACCC2038, AS2.311, AS2.259, ACCC2045, IFFI1036, AS2.371, AS2.559, AS2.440 and AS2.704.

6. The composition of claim 1, wherein said composition is in the form of a tablet, powder, or a health drink.

7. The composition of claim 6, wherein said composition is in the form of a health drink.

8. A method for treating hyperlipemia in a subject comprising administering to said subject a composition of claim 1.

9. The method of claim 8 comprising oral administration.

10. A method of preparing a yeast composition, comprising culturing a plurality of yeast cells in the presence of an alternating electric field having a frequency in the range of about 7000 to 13000 MHz and a field strength in the range of about 200 to 450 mV/cm, wherein said composition is capable of treating hyperlipemia in a subject.

11. The method of claim 10, wherein said frequency is in the range of about 7500-8000, 10000-10500, or 12400-12800 MHz.
